(19) United States
(12) Patent Application Publication
(10) Pub. No.: US 2017/0258635 A1
(43) Pub. Date: Sep. 14, 2017

(54) METHOD AND DEVICE FOR ELECTROLYSIS OF AQUEOUS HUMOR TO TREAT GLAUCOMA

(71) Applicant: Michael REYNARD, Santa Monica, CA (US)

(72) Inventor: Michael REYNARD, Santa Monica, CA (US)

(21) Appl. No.: 15/457,724

(22) Filed: Mar. 13, 2017

Related U.S. Application Data
(60) Provisional application No. 62/307,623, filed on Mar. 14, 2016.

(51) Int. Cl. A61F 9/007; A61N 1/08; H01M 10/46; H01M 10/0525; H01M 10/34; H01M 10/30; A61N 1/05; H01M 6/04

(52) U.S. Cl. CPC A61F 9/00781; A61N 1/0543; A61N 1/08; H01M 6/045; H01M 10/0525; H01M 10/345; H01M 10/30; H01M 10/46; A61F 2250/0001; H01M 2220/30

(57) ABSTRACT

A glaucoma treatment device applies electrolysis into an eye has a plurality of electrodes connected to a voltage source, and a controller coupled to a pressure sensor. The electrodes apply an electric field within an eye, and the controller regulates the delivery of current to the electrodes based on intraocular pressure measurements from the pressure sensor. The device has an enclosure for the pressure sensor, controller, and voltage source. The voltage source can recharge via an external source and the controller can accept adjustments remotely. The device also operates as a component in a method to convert aqueous humor into gas. The method utilizes electrolysis to reduce the volume of fluid in the anterior chamber of an eye. The method modulates electric current during usage of the invention. The method and device combine to reduce intraocular pressure within an eye thus lessening the progression of glaucoma.

### CROSS-REFERENCE TO RELATED APPLICATION

This non-provisional application claims priority to pending provisional application No. 62/307,623 filed on Mar. 14, 2016, all of which are owned by the same inventor.

### BACKGROUND OF THE INVENTION

The present invention relates generally to treatment of glaucoma and more particularly to a method and its device of converting aqueous humor into gases by means of applying an electric field to reduce intraocular pressure, and evacuating the gases from an eye.

Glaucoma is a potentially blinding optic neuropathy and is the second most common cause of blindness in the world. Glaucoma affects one in 200 people under the age of fifty. Optic nerve damage caused by glaucoma is generally associated with elevated intraocular pressure, primarily by obstruction of the outflow of aqueous humor from the eye, or less frequently, by excess production of aqueous humor within the eye. In primary open angle glaucoma, the most common form of glaucoma, aqueous humor outflow is impaired through the trabecular meshwork. In closed angle glaucoma, the peripheral iris blocks access of aqueous humor to the trabecular meshwork. In either situation, the accumulation of aqueous humor results in elevated intraocular pressure.

Aqueous humor is a clear fluid produced by the non-pigmented cells of the ciliary body that forms the anterior and posterior chambers of an eye to keep the eyeball inflated. The aqueous humor provides nutrition to ocular tissue, removes excretory products, and transports neurotransmitters. The major components of the aqueous humor are organic and inorganic ions, proteins, amino acids, carbohydrates, glutathione, urea, oxygen, carbon dioxide and water. The ionic concentration of the aqueous humor facilitates electrical conductivity. The anterior chamber of the human eye has a volume of 250 microliters and the posterior chamber has a volume of 60 microliters. The rate of aqueous humor production is between 2 to 3 microliters per minute. At this rate of production, the turnover of aqueous humor is 1.0% to 1.5% of the anterior chamber volume per minute.

Most treatments for glaucoma involve reducing intraocular pressure by reducing the production of aqueous humor or by enhancing the outflow of aqueous humor. Topically applied medications such as adrenergic agonists or beta blockers reduce intraocular pressure by decreasing aqueous production. Selective laser trabeculoplasty may enhance outflow by targeting intracellular melanin granules in the trabecular meshwork. A variety of glaucoma drainage devices serve as shunts for transmitting aqueous humor from the anterior chamber to the exterior of the eye. Redirection of aqueous fluid from the anterior chamber to the exterior of the eye has the effect of reducing intraocular pressure.

A variety of tube shunts are available for diverting aqueous humor to an external reservoir that is situated on the surface of the eyeball. The aqueous fluid in the reservoir is absorbed in the veins and in the general circulation. Certain tube shunts have a valve that limits the flow of aqueous in one direction. The valve helps prevent the pressure in the eye from becoming too low. Filtration surgery (trabeculectomy) for glaucoma creates a fistula from the anterior chamber to the surface of the eyeball. Filtration surgery and glaucoma drainage shunts release aqueous humor into the subconjunctival space. Seepage of aqueous humor in the subconjunctival space forms a pocket of fluid called a bleb. Other forms of treatment have included physical or thermal destruction of the ciliary body such as cyclodiathermy or cycloelectrolysis, and the use of shunts such as the Ahmed Glaucoma implant.

Destruction of the ciliary body and shunts to treat glaucoma has multiple disadvantages. Reduction of aqueous humor production by destruction of the ciliary body is not predictable. As a result, excessive destruction may result in ocular hypotony. Medications to treat glaucoma have a prohibitive cost for most glaucoma patients. Selective laser trabeculoplasty is limited by its ability to reduce intraocular pressure by only a few points, and is not readily available for many glaucoma patients because of its expense. Additionally, glaucoma drainage shunts are complicated to perform and expensive. Blebs formed by filtration surgery or glaucoma valves are prone to failure because of scar tissue. Blebs can also become infected and lead to endophthalmitis with loss of vision. Glaucoma drainage shunts that divert aqueous humor tend to clog over time and may also become malpositioned. Clogging of the shunt may render it ineffective and a malpositioned valve may cause damage to the cornea and other intraocular structures.

In view of the limited effectiveness of treatment options, therefore there is a need to develop more effective treatments for glaucoma. The present invention provides a method and device for converting aqueous humor to gas by means of an electric field in the anterior chamber or in the lumen of a glaucoma shunt. Reduced volume of aqueous humor caused by electrolytic conversion results in reduced intraocular pressure. Electrolysis of aqueous humor also eliminates the need for a bleb and its associated complications.

### DESCRIPTION OF THE PRIOR ART

Ophthalmologists and others have treated glaucoma using various methods. Some methods curtail production of aqueous humor thus reducing the volume of the humor within an eye and hence the pressure. Other methods install a shunt that drains the humor more rapidly from within the eye. Still other methods surgically alter the drainage structures of an eye.

In a related area, Blum in U.S. Pat. Pub. No. 2012/0140167, teaches of a dynamic chargeable contact and intraocular lens that includes an electronic component. The electronic component comprises an electromagnet that activates the dynamic optic. In some embodiments the power generator has a coupling to a rechargeable battery capable of charging remotely by radio frequency excitation. Blum also adjust the shape, and hence, the power of the lens.

Modules for data transmission exist in the prior art as well. For example, Ohaynn et al in U.S. Pat. No. 8,964,646 teaches of a virtual broadband transmitting unit that generates data streams to a wireless communication network. Ohayn describes a plurality of modems and a modem manager for sending data packets wirelessly into the Internet. Ohyan utilize a satellite communications network for transmissions from select modems.

### SUMMARY OF THE INVENTION

The present invention is directed to electrolytic conversion of aqueous humor to hydrogen and oxygen gases as a means to treat glaucoma. A glaucoma treatment device applies electrolysis into an eye by means of a plurality of electrodes connected to a voltage source, and a controller coupled to a pressure sensor. The electrodes apply an electric field within an eye, and the controller regulates the delivery of current to the electrodes based on intraocular pressure measurements from the pressure sensor. The device operates as a component in a method to convert aqueous humor to hydrogen and oxygen gases by electrolysis. The method utilizes electrolysis to reduce the volume of aqueous humor fluid in the anterior chamber of an eye. The method and device combine to reduce intraocular pressure within an eye thus lessening the progression of glaucoma.

In one embodiment consistent with the principles of the present invention, a method of treating glaucoma by applying an electric field within the anterior chamber of an eye results in electrolytic conversion of aqueous humor into hydrogen and oxygen. The ionic content of aqueous humor facilitates conductivity between the cathode and anode. The gases emitted through electrolysis are absorbed by blood and ocular tissue. During this process, the concentration of gases in the anterior chamber is reduced as it diffuses according to Fick's first law of diffusion where a solute will move from a region of high concentration to a region of low concentration across a concentration gradient. Fick's law of diffusion relates the concentration of gas produced by electrolysis to the environment. In a mammalian eye, the environment consists of blood, outer wall of ocular tissue, and aqueous humor. The dissipation of gas produced by electrolysis of aqueous is dependent on surface area of each environmental component, i.e. thickness of blood vessel walls and the thickness of outer wall of ocular tissue. In addition, some of the gas will become reabsorbed into the aqueous. Electrolysis of aqueous humor is accomplished by means of a pair of electrodes inserted into the anterior chamber. Electrodes, coupled to a voltage source of sufficient power, cause electrolysis and conversion of aqueous humor into elemental gases. There has thus been outlined, rather broadly, the more important features of the invention in order that the detailed description thereof that follows may be better understood and that the present contribution to the art may be better appreciated. The other embodiments and additional features of the invention will be described hereinafter and which will form the subject matter of the claims attached.

In another embodiment, the present invention is a device for applying an electric field within the fluid pathway of a glaucoma shunt. The device has a pair of electrodes configured to apply an electric field within the lumen of the glaucoma shunt. The pair of electrodes is coupled to a voltage source. A pressure sensor that measures intraocular pressure is coupled to a controller. The controller applies the electric field to aqueous humor within the lumen of a glaucoma shunt. The controller uses intraocular pressure readings from the pressure sensor to control the applied electric field. When intraocular pressure is elevated, input to the controller results in a higher level of voltage or frequency of voltage delivery to the aqueous humor. The volume of gas released from electrolysis and absorbed by ocular tissue is balanced against the production of aqueous humor.

In another embodiment, the present invention has a module for data transmission that provides wireless transmission of information provided by the pressure sensor. Information retained by the module for data transmission can include intraocular pressure readings based on time, and a warning message for low battery voltage levels.

Numerous objects, features and advantages of the present invention will be readily apparent to those of ordinary skill in the art upon a reading of the following detailed description of the presently preferred, but nonetheless illustrative, embodiment of the present invention when taken in conjunction with the accompanying drawings. Before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, the phraseology and terminology employed herein are for the purpose of description and should not be regarded as limiting.

One object of the present invention is to provide a method and device for electrolytic conversion of aqueous humor to gas as a means to lower intraocular pressure as treatment for glaucoma.

Another object is to provide such a method and device for electrolytic conversion of aqueous humor to treat glaucoma that also assists in detecting the level of intraocular pressure.

Another object is to provide such a method and device for electrolytic conversion of aqueous humor to treat glaucoma that has low cost of manufacturing so the surgeons, practices, clinics, hospitals, vendors, and technicians can readily acquire the invention through supply houses, catalogs, preferred vendors, and select manufacturers.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated a preferred embodiment of the invention.

### BRIEF DESCRIPTION OF THE DRAWINGS

In referring to the drawings,

FIG. 1 provides a cross-sectional diagram of the general anatomy of the anterior segment of a mammalian eye;

FIG. 2 provides a cross-sectional diagram of the anterior segment of a mammalian eye showing aqueous flow into and through its anterior chamber;

FIG. 3 provides a schematic view of the front portion of an eye having the invention thereon;

FIG. 4 provides a block diagram according to the principles of the present invention;

FIG. 5 provides a cross sectional view of the invention when implanted in an eye;

FIG. 6 provides a schematic view of an alternate embodiment of the invention with suture holes for securing it to the sclera of an eye; and,

FIG. 7 illustrates a further alternate embodiment of the invention when used with a conventional glaucoma drainage device.

The same reference numerals refer to the same parts throughout the various figures.

### DESCRIPTION OF THE PREFERRED EMBODIMENT

The present invention overcomes the prior art limitations by providing a method and device for electrolytic conversion of aqueous humor to treat glaucoma. This method and its device minimize permanent damage to the structures of an eye while relieving the high intraocular pressure from the eye characteristic of glaucoma.

Referring to FIG. 1, relevant structures of a mammalian eye appear in this figure and provide background information for the anatomical terms used herein. Though this description refers to mammalian eye, the Applicant foresees both human medicine and veterinary medicine applications for the invention. Though this description refers to mammalian eye and eye, that description includes both human and non-human mammal eyes. FIG. 1 provides a cross sectional view of an anterior segment E of the eye. As shown in FIG. 1 towards the upper center, the cornea C is a thin transparent tissue which is part of the outer eye and lies in front of an iris I. The iris includes a pupil P as an opening for the passage of light into the eye for vision. The cornea merges into a sclera M at a juncture known as a limbus Q here shown towards the right. The sclera has an interior layer called the choroid J that extends around the eye but does not cover the interior surface of the lens. A layer of tissue called the bulbar conjunctiva, later shown in FIG. 5 covers the exterior of the sclera M. A ciliary body D extends from an uveal tract (not shown) beginning on the left of the figure. It begins at the limbus Q and extends along the interior of the sclera M to the right ending at the uveal tract K. The uveal tract includes the ciliary body, the iris I, and the choroid. A crystalline lens L of the eye is situated between the iris I and a vitreous fluid F and remains enclosed within a transparent lens capsule as at H. An anterior chamber G is the space between the cornea C and the crystalline lens L, here shown above and outwardly from the lens. A posterior chamber, as at G′, is a space bounded by the iris, ciliary body, and crystalline lens. Both of the anterior and posterior chambers receive aqueous humor B fluid within them to fill them. A trabecular meshwork A here shown outwardly from the lens L at the sclera M, filters aqueous humor from the anterior chamber into drainage channels (not shown), surrounding capillaries (not shown), and lymphatic ducts of the lymphatic system (not shown). Proximate the vicinity of the cornea C and the iris I, the eye has an angle R. The angle R is a portion of the anterior chamber and is filled with aqueous humor B along with the remainder of the anterior chamber. The base of the angle R has the trabecular meshwork A and through there the aqueous humor drains B into microscopic pores (not shown) in the trabecular meshwork and into the general blood circulation/collector channels (not shown). In open angle glaucoma the pores of the trabecular meshwork A have defects and obstruct the flow of aqueous humor. In closed angle glaucoma, the iris comes into contact with the cornea to prevent passage of aqueous humor into the trabecular meshwork. In both forms of glaucoma, the pressure of the eye increases as a result of build-up of the aqueous humor fluid.

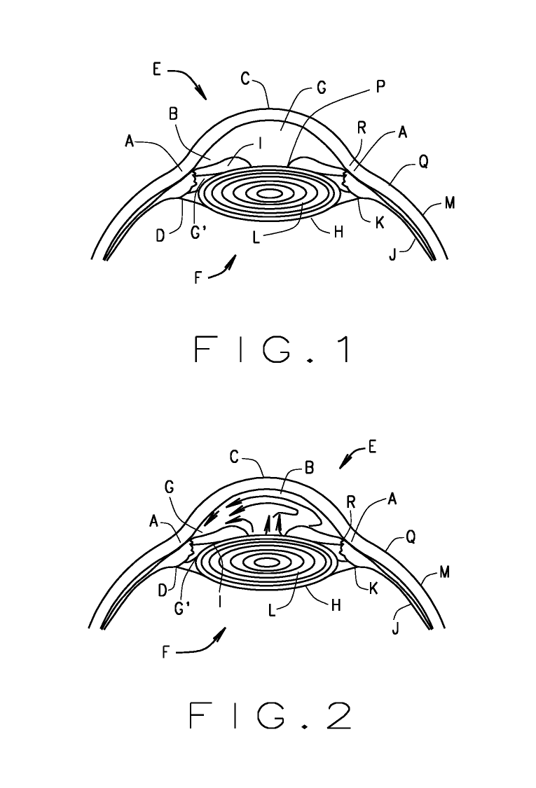

As shown, FIG. 2 provides a cross sectional view of an anterior segment E of the human eye. Here in FIG. 2, the aqueous humor B flows, as shown, from the posterior chamber G′, through the pupil, into the anterior chamber G. The aqueous humor flows from the interior to the exterior of the eye.

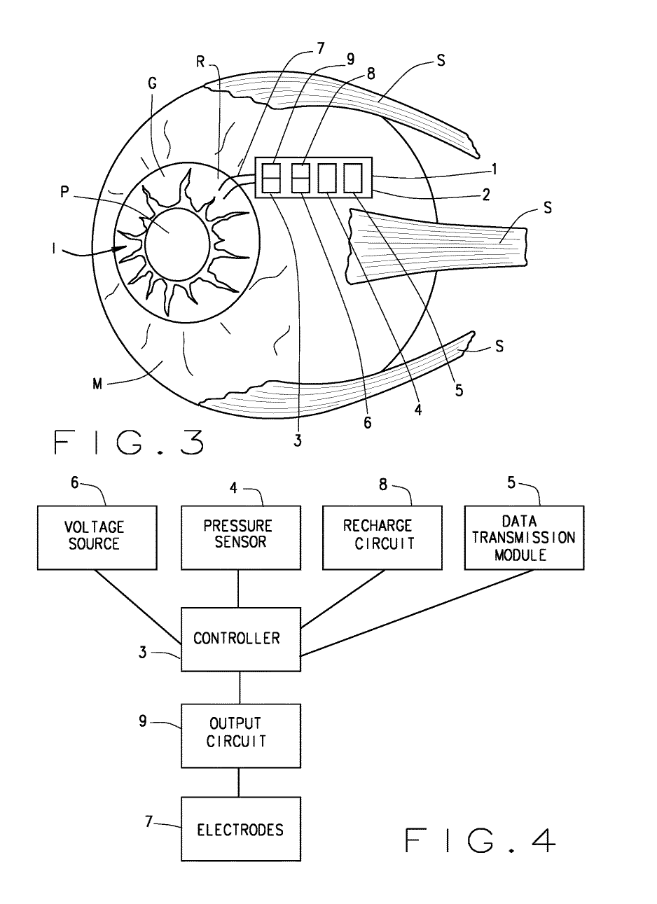

FIG. 3 provides a schematic view of the front portion of an eye with the invention 1 installed thereon. The eye has its classic round, nearly spherical shape as shown with the pupil P and iris I to the left of this figure. Outwardly from the pupil, the anterior chamber G has a generally centered position upon the eyeball and the sclera M extends and enwraps the remainder of the eyeball. This figure shows three muscles S, among others, that orient the eyeball for vision. These muscles are shown attached at the top, the bottom, and the left middle of the eyeball.

Then FIG. 3 illustrates a method of applying electrolysis, or electrolyzing, formerly vaporizing, aqueous humor of the present invention. FIG. 3 then shows the invention 1 of an implant with an enclosure 2 positioned, or installed, on the sclera M containing a controller 3, a pressure sensor 4, a data transmission module 5, and voltage source 6, or a power source, such as a battery, capacitor, or photocell. The battery may have composition using alkaline, silver nitrate, nickel cadmium, nickel metal hydride, and the like. The battery may also have a recharging capability from its composition using lithium ion, lithium polymer, and a photocell in communication with a capacitor.

The enclosure has a preferable form of a plastic polymer, such as a silicone elastomer. The enclosure may contain a radiopaque material such as barium sulfate, to make the implant visible in x-ray procedures. The enclosure has a radius of curvature no less than 12 mm and preferably in the range of about 12 mm to about 15 mm. The length of the enclosure is no more than 13 mm and preferably about 10 mm and its width is no more than 13 mm and preferably about 6 mm. The inner surface of the enclosure is flat or preferably concave so as to conform to the curvature of the eye. The invention installs upon an eye so that the conjunctiva covers the electrodes and the enclosure, as later shown in FIG. 5. Electrodes 7, here shown at least two in number, that is, as a pair extending from the enclosure, enter the sclera posterior to the limbus and have a position within the angle R of the anterior chamber. The electrodes enter the anterior chamber of a mammalian eye. The electrodes have sufficient length to span the thickness of the cornea and the sclera. Alternatively, the electrodes have sufficient length to span solely the cornea or solely the sclera. Alternatively the electrodes have a position through a surgically constructed scleral tunnel before entering the angle of the anterior chamber. The electric field between the pair of electrodes causes electrolysis of aqueous humor and its conversion into hydrogen and oxygen gases. The surrounding tissue then absorbs the gases, including the sclera. Also, the electrolytic conversion of aqueous humor to gases results in reduced fluid volume and reduced intraocular pressure. The electrodes span the thickness of the sclera and the encasing conjunctiva. Thus, the electrodes have a length between about 250 microns to about 750 microns. The electrodes connect to the voltage source 6 and the controller 3 situated within the enclosure 2 that optionally contains the pressure sensor 4. The present invention has a size of approximately 12 millimeters by 12 millimeters and a thickness compatible with implantation on the surface of an eye and secured by sutures and/or tissue adhesive. The present invention has its electrodes 7 extending outwardly from it. The electrodes preferably have a coupling to the enclosure 2 by a flexible lead, or alternatively by a rigid lead. The electrodes may also serve as prongs that stabilize the invention in position.

The present invention can be implanted using known ophthalmological surgical techniques and, with reference to FIG. 3 and FIG. 5, a brief discussion of the surgical implant procedure follows. The surgeon makes an initial incision in the conjunctiva posterior to the limbus. The enclosure is inserted through this incision and positioned on the sclera. Eyelets, in the forefront of the enclosure, are used to suture the enclosure to the sclera. The electrodes are inserted through the sclera posterior to the limbus and into the angle of the anterior chamber. Alternatively, a surgically constructed scleral tunnel may serve as a protective conduit for electrodes which are then surgically guided into the angle of the anterior chamber. The exposed portion of the enclosure or electrode is covered with graft material consisting of sclera, amniotic tissue, or pericardium.

FIG. 4 provides a block diagram of the invention according to the principles of the present invention. With the enclosure 2, the invention 1 has its voltage source 6, the pressure sensor 4 as an optional component, a circuit for recharge as at 8, the controller 3, an output circuit 9, and the pair of electrodes 7. The recharge circuit in cooperation with the transmission module receives an external signal and converts it to electrical power for delivery to the power source. The voltage source has electrical communication to the controller which distributes power to the remainder of the invention. The pressure sensor, the recharge circuit, and the data transmission module provide data input to the controller. The controller then outputs, or metes, a regulated amount of voltage through the output circuit 9 which then energizes the electrodes 7 at the appropriate strength for the opportune time.

FIG. 5 provides a cross sectional view of the invention when implanted in an eye, particularly upon the sclera M and beneath the conjunctiva N as shown. The sealed enclosure 2 contains some or all of the previously described components. Aqueous humor B produced by the ciliary body as a portion of K flows into the anterior chamber G beneath the cornea C. The enclosure is implanted beneath the conjunctiva N posterior to the limbus L. Electrodes 7 exit the enclosure then enter the anterior chamber G through the limbus L, where they contact the aqueous humor.

The electrodes conduct charge and may use a variety of materials, including but not limited to titanium, nickel titanium, brass, nickel, aluminum, platinum, iridium, iridium oxide, titanium nitride, tantalum, stainless steel, other steels, graphite, alloys or combinations thereof. In an alternate embodiment, the electrodes are either titanium or platinum. The electrode may also have composite construction, whereby different sections have a construction from different materials. In the preferred embodiment, the electrodes have a medical grade material safe for prolonged use inside an eye. The electrodes are embedded in a non-conductive sheath that serves as an insulating barrier so other areas of the eye are not affected by the electric field. The insulating sheath preferably has sufficient thickness to prevent both current flow and capacitance coupling with the tissue.

The electrodes may have various shapes such as straight, angles or curved, which provide for an optimal electric field while avoiding contact with intraocular tissue. The configuration and location of the electrodes have optimal form to maximize the effectiveness of the electric field in aqueous humor while minimizing the impact on surrounding tissue. As mentioned above, voltage applied across the electrodes 7 creates an electric field. The typical electric field strength is between about 1 to 25,000 Volts per centimeter, and preferably about 1 to about 250 Volts per centimeter.

The controller 3 regulates the voltage applied to the electrodes which may have continuous or intermittent form, that is, the controller metes power to the electrodes. The controller and voltage source may have the form of a single device. The electric field generated by the electrodes results in electrolytic conversion of aqueous humor to hydrogen and oxygen gases as noted above. The spacing of each electrode in the pair ranges from about 100 to about 1000 microns. A close spacing of electrodes permits high levels of electrolysis with the use of moderate voltages and minimal power consumption than a wider spacing.

The shape of the electrodes 7 may take many forms. A preferred embodiment of electrodes though has sharp tips upon the electrodes so that they perforate the sclera during usage. Electrodes with sharp tips enhance electrolytic conversion of aqueous humor to gases. Piercing the sclera for placement of electrodes in the anterior chamber occurs more readily using electrodes having sharp tips. Though the electrodes have tips, the tips may have a round conical shape, a beveled shape, an arrowhead shape, a narrow pin, and the like.

A plurality of sources may provide voltage to the invention, as at 6. The voltage source 6 includes a rechargeable battery, such as a lithium ion or lithium polymer, a photocell, or a capacitor. A voltage source can be recharged by a radio-frequency identification, or “RFID,” link or other type of electric recharging circuit. The surgeon selects the voltage source 6 based upon available space, amount of current needed, lifespan of the device, cost, installation methods, and the like. The voltage source supplies charge to the electric field between the electrodes generally between about 1 to about 500 volts, and preferably about 1 to about 250 volts.

The pressure sensor 4 has a location upon the sclera M so that it monitors intraocular pressure and provides feedback to the controller. The Applicant foresees deploying a pressure sensor as a component of the invention so that it monitors intraocular pressure before, during, and after usage of the invention. A controller operating in concert with the pressure sensor can regulate the amount of voltage passing to the electrodes. The pressure sensor acts in concert with the power source and controller to provide current only when needed. The controller has a program that releases electrical power to the electrodes only when the pressure reaches a certain threshold. The controller accepts adjustments to its program remotely to adjust for a desired pressure threshold. In an alternate embodiment, the Applicant also foresees combining a temperature sensor with the pressure sensor. The temperature sensor serves as a fall back to the pressure sensor and acts in concert with the power source and controller to restrict current when no longer needed. The controller has a program that ceases delivery of electrical power to the electrodes when the temperature exceeds a certain value, typically about 95° C. at about sea level pressure, and lower temperatures at lower pressures typically at higher altitudes.

The controller can also modulate the duration of electric current reaching the electrodes. A constant duration of current produces constant electrolysis of aqueous humor while, intermittent current produces an electrolysis used only when the level of pressure rises sufficiently to warrant electrolysis of aqueous humor. The duration of current and the voltage level vary by the controller to regulate the intensity of electrolysis of aqueous humor. Preferably, the current supplied in the invention for electrolysis of aqueous humor is not continuous. This type of current markedly reduces the volume of battery.

More particularly, the controller provides the invention with a voltage of an amplitude between about 0.1 volt and about 60 volts. The controller imparts into the invention, through the output circuit, electrical stimulation of a current having a pulse amplitude of between about 10 microamps to about 25 milliamps. The stimulation also has a current having a pulse width between about 50 microseconds to about 2700 microseconds. The controller also has a voltage limiting circuit that limits the voltage emitted by the stimulation component during usage.

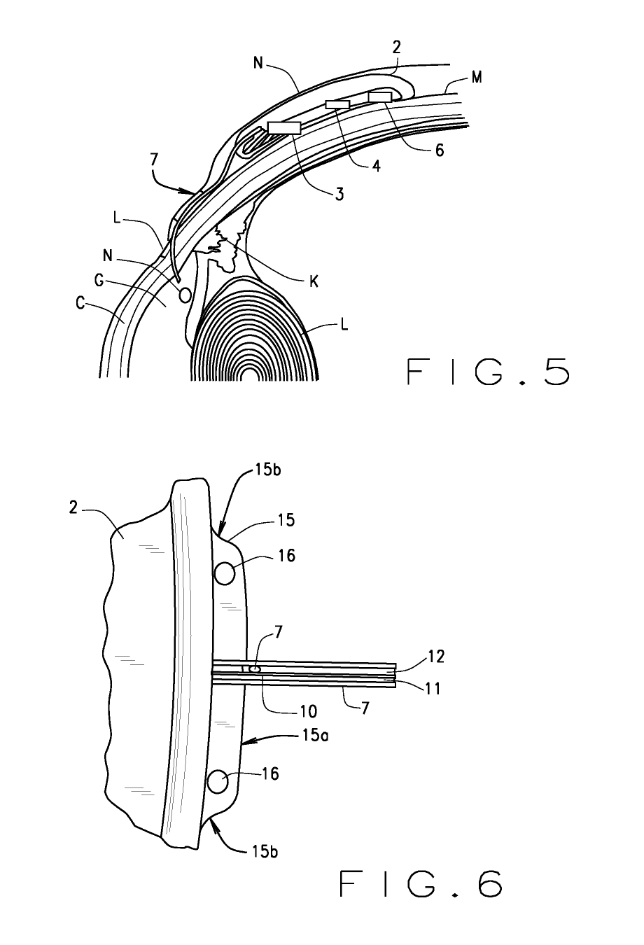

FIG. 6 provides a schematic view showing an alternative embodiment of the invention with suture holes for securing the invention to the sclera. The suture holes appear upon an extension 15 of the inner portion of the enclosure 2 that includes a plurality of small eyelets 16, here shown as two. The extension 15 has a generally rectangular form with a longitudinal edge, 15a, outwardly from the enclosure and two spaced apart lateral edges, 15b, spanning from the longitudinal edge to the enclosure. The lateral edges have a beveled appearance in this figure. The lateral edges have much less length than the longitudinal edge as shown. The eyelets 16 serve as suture holes that a surgeon uses to secure the invention to the sclera. The surgeon loops suture material, such as nylon, through the eyelets and secures them by attachment to the sclera.

Generally centered upon the extension 8, an electrode 7 extends outwardly from the extension and the remainder of the enclosure 2. Each electrode has a sheath 10 of a non-conductive, insulating material. The insulating material spaces two adjacent electrodes a particular distance apart. The insulating material also prevents electrical discharge to intraocular structures that are not targeted for electrolysis. Further, the insulating material isolates an anode 11 portion of the electrode from the cathode 12 portion of the electrode.

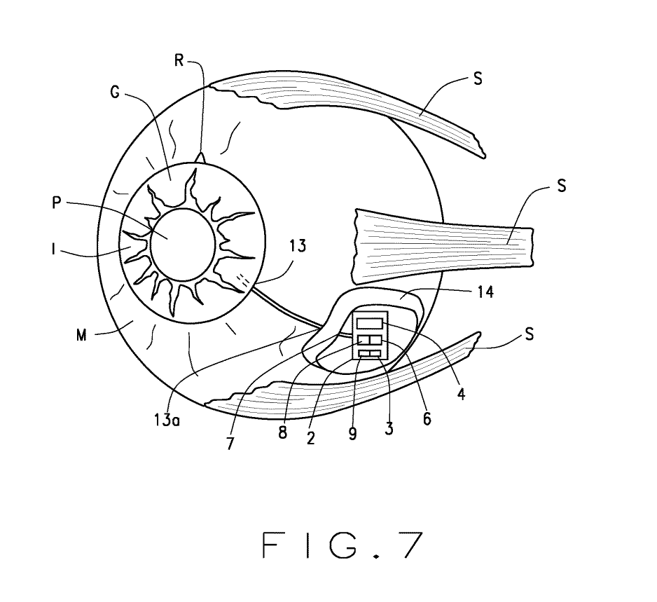

FIG. 7 illustrates a further embodiment of the invention when used with a conventional glaucoma drainage device. FIG. 7 also shows a perspective view of a tubular glaucoma drainage shunt and the invention 1 mounted upon the exterior surface of an eye ball, or sclera M. The shunt has fluidic communication as at 13 that enters the anterior chamber. Electrodes 7, extending from the enclosure 2 of the invention 1, penetrate the outer shell of the glaucoma shunt as at 13a and into the invention's lumen as at 14. The electrodes 7 also mechanically stabilize the position of the enclosure 2 on the exterior surface of a glaucoma valve, not shown. The electrodes, present in the lumen of the glaucoma shunt, provide an electric field for electrolysis of aqueous fluid. The gasses from electrolysis dissipate through the channels for aqueous humor outflow, normally present in glaucoma shunts. The enclosure 2 of the invention contains the voltage source 6 and the other components of the invention as previously described. The components also include the pressure sensor 4 that provides information to the controller. The controller then maintains equilibrium between the intraocular pressure in an eye and the degree of electrolysis.

The present invention can be implanted on the surface of a glaucoma drainage shunt using known ophthalmological surgical techniques. Referring to FIG. 7, the Applicant describes the surgical implant procedure suitable for the invention. The surgeon retracts the conjunctiva, or incises it, and then inserts the enclosure 2 inserted through this opening. Electrodes 7, exiting the enclosure 2, then penetrate the top of a glaucoma drainage shunt under the surgeon's guidance and enter the lumen of the shunt. The surgeon penetrates the shunt by drilling the roof of the glaucoma drainage shunt with a small drill bit or by piercing the top surface of the glaucoma drainage device with sharp-tipped electrodes. The electrodes also mount the enclosure securely in position upon the outer shell, or lumen 14, of the glaucoma shunt. Electrolysis of aqueous humor within the lumen of the glaucoma drainage eliminates the cause for the formation of a bleb and its potential complications.

The device of the invention permits a surgeon to utilize this method to thwart the progression of glaucoma: reducing intraocular pressure in a mammalian eye. The method provides at least two electrodes and a lead extending from each electrode, provides a controller receiving the leads and having an output circuit in communication with the leads to the two electrodes so the controller receives external communications through a transmission module, providing a power source in communication with the controller so it metes power into the leads of the electrodes, provides a recharge circuit that receives an external signal and generates electrical power for the power source, placing the controller, the power source, the output circuit, and the recharge circuit within an enclosure. The method installs the enclosure upon the sclera of a mammalian eye, inserts the two electrodes into the anterior chamber of a mammalian eye, regulates the spacing of the two electrodes and the field strength generated by them, regulates the voltage, current, and duration of electrical power application by the controller through the output circuit for dispensing into the electrodes. The method has its goal of upon application of electrical power to the electrodes a portion of aqueous humor electrolyzes and exits a mammalian eye thus lowering its pressure.

From the aforementioned description, a method and device for electrolysis of aqueous humor to treat glaucoma has been described. The method and device for electrolysis of aqueous humor to treat glaucoma is uniquely capable of converting aqueous humor into its constituent gases using intermittent electrical current until intraocular pressure falls below a threshold value. The method and device for electrolysis of aqueous humor to treat glaucoma and its various components may be manufactured from many materials, including but not limited to, vinyl, polymers, such as nylon, polypropylene, polyvinyl chloride, high density polyethylene, polypropylene, ferrous and non-ferrous metal foils, their alloys, and composites.

Various aspects of the illustrative embodiments have been described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations have been set forth in order to provide a thorough understanding of the illustrative embodiments. However, it will be apparent to one skilled in the art that the present invention may be practiced without the specific details. In other instances, well known features are omitted or simplified in order not to obscure the illustrative embodiments.

Various operations have been described as multiple discrete operations, in a manner that is most helpful in understanding the present invention, however, the order of description should not be construed as to imply that these operations are necessarily order dependent. In particular, these operations need not be performed in the order of presentation.

Moreover, in the specification and the following claims, the terms “first,” “second,” “third” and the like—when they appear—are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to ascertain the nature of the technical disclosure. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

As such, those skilled in the art will appreciate that the conception, upon which this disclosure is based, may readily be utilized as a basis for the designing of other structures, methods and systems for carrying out the several purposes of the present invention. Therefore, the claims include such equivalent constructions insofar as they do not depart from the spirit and the scope of the present invention.

I claim:

1. An electrolysis device, comprising:
at least two electrodes, a lead extending from each electrode;
a controller receiving said leads, said controller having an output circuit in communication with said leads to said at least two electrodes;
said controller receiving external communications through a transmission module;
a power source in communication with said controller, wherein said controller metes power into said leads and thus to said at least two electrodes;
a recharge circuit adapted to receive an external signal and generate electrical power for said power source; and,
an enclosure containing said controller, said power source, said output circuit, and said recharge circuit within it;
wherein said enclosure is adapted to install upon the sclera of a mammalian eye and said at least two electrodes are adapted to enter the anterior chamber of a mammalian eye.

2. The electrolysis device of claim 1, further comprising:
said recharge circuit receiving a signal from said transmission module wherein said recharge circuit converts the signal into electrical power for delivery to said power source.

3. The electrolysis device of claim 1 wherein said power source is one of a battery and a rechargeable battery; and,
said battery is one of alkaline, silver nitrate, nickel cadmium, and nickel metal hydride; and,
said rechargeable battery is one of lithium ion, lithium polymer, and a photocell in communication with a capacitor.

4. The electrolysis device of claim 1 wherein said at least two electrodes have a mutual spacing of about 100 microns to about 1000 microns and an electric field strength of about 1 volt per centimeter to about 25,000 volts per centimeter.

5. The electrolysis device of claim 1 wherein said controller dispenses through said output circuit a voltage from about 1 volt to about 500 volts and a current from about 10 microamps to about 25 milliamps for a time of application from about 50 microseconds to about 2700 microseconds.

6. The electrolysis device of claim 1, further comprising:
said enclosure having a width no more than 13 millimeters and a radius of curvature no less than 12 millimeters.

7. The electrolysis device of claim 2 further comprising said electrodes being one of titanium, nickel titanium, brass, nickel, aluminum, platinum, iridium, iridium oxide, titanium nitride, tantalum, stainless steel, and graphite.

8. The electrolysis device of claim 2 further comprising:
two electrodes of one of titanium and platinum.

9. The electrolysis device of claim 1 further comprising:
a pressure sensor and a temperature sensor, said pressure sensor and said temperature sensor locating within said enclosure;
said controller receiving input from said pressure sensor and from said temperature sensor; and,
wherein said electrolysis device is adapted to communicate with a glaucoma shunt.

10. A method of reducing intraocular pressure in a mammalian eye, comprising:
providing at least two electrodes and a lead extending from each electrode;
providing a controller receiving said leads and having an output circuit in communication with said leads to said at least two electrodes wherein said controller receives external communications through a transmission module;
providing a power source in communication with said controller, wherein said controller metes power into said leads and to said at least two electrodes;
providing a recharge circuit adapted to receive an external signal and generate electrical power for said power source; and,
placing said controller, said power source, said output circuit, and said recharge circuit within an enclosure;
installing said enclosure upon the sclera of a mammalian eye;
inserting said at least two electrodes into the anterior chamber of a mammalian eye;
regulating the spacing of said at least two electrodes and the field strength generated by said at least two electrodes; and,
regulating the voltage, current, and time of electrical power by said controller through said output circuit for dispensing into said at least two electrodes;
wherein upon application of electrical power to said at least two electrodes a portion of aqueous humor electrolyzes and exits a mammalian eye thus lowering its pressure.

11. The method of reducing intraocular pressure in a mammalian eye of claim 10 further comprising:
said regulating the voltage, current, and time of electrical power by said controller dispenses through said output circuit a voltage from about 0.1 volts to about 500 volts and a current from about 10 microamps to about 25 milliamps for a time of application from about 50 microseconds to about 2700 microseconds.

12. The method of reducing intraocular pressure in a mammalian eye of claim 11 further comprising:
said regulating the voltage, current, and time of electrical power by said controller dispenses through said output circuit a voltage from about 0.1 volts to about 60 volts.

13. The method of reducing intraocular pressure in a mammalian eye of claim 10 wherein said regulating the spacing and the field strength of said at least two electrodes includes a mutual spacing of said at least two electrodes about 100 microns to about 1000 microns and an electric field strength of about 1 volt per centimeter to about 25,000 volts per centimeter.

14. The method of reducing intraocular pressure in a mammalian eye of claim 13 wherein said regulating the voltage, current, and time of electrical power by said controller dispenses through said output circuit a voltage from about 0.1 volts to about 60 volts and a current from about 10 microamps to about 25 milliamps for a time of application from about 50 microseconds to about 2700 microseconds;
wherein said regulating the spacing and the field strength of said at least two electrodes includes a mutual spacing of said at least two electrodes about 100 microns to about 1000 microns and an electric field strength of about 20 volts per centimeter to about 5000 volts per centimeter; and,
wherein said method of reducing intraocular pressure in a mammalian eye is adapted to communicate into a glaucoma shunt.

15. The method of reducing intraocular pressure in a mammalian eye of claim 10 further comprising:
said providing a recharge circuit receiving a signal from said transmission module converting the signal into electrical power for delivery to said power source.

16. The method of reducing intraocular pressure in a mammalian eye of claim 10 further comprising:
said providing a power source including one of a battery and a rechargeable battery;
wherein said battery is one of alkaline, silver nitrate, nickel cadmium, and nickel metal hydride; and,
wherein said rechargeable battery is one of lithium ion, lithium polymer, and a photocell in communication with a capacitor.

17. The method of reducing intraocular pressure in a mammalian eye of claim 16 further comprising:
said providing at least two electrodes, said at least two electrodes being one of titanium, nickel titanium, brass, nickel, aluminum, platinum, iridium, iridium oxide, titanium nitride, tantalum, stainless steel, and graphite.

18. The method of reducing intraocular pressure in a mammalian eye of claim 10 further comprising:
providing a pressure sensor in communication said controller;
providing a temperature sensor in communication said controller; and,
ceasing delivery of current by said controller through said output circuit to said at least two electrodes upon said temperature sensor detecting about 95° C. at about sea level pressure.

19. An electrolysis device, comprising:
at least two electrodes, a lead extending from each electrode, said at least two electrodes have a mutual spacing of about 100 microns to about 1000 microns and an electric field strength of about 1 volt per centimeter to about 25,000 volts per centimeter, said at least two electrodes being one of titanium, nickel titanium, brass, nickel, aluminum, platinum, iridium, iridium oxide, titanium nitride, tantalum, stainless steel, and graphite;
a controller receiving said leads, said controller having an output circuit in communication with said leads to said at least two electrodes;
said controller receiving input from a pressure sensor and a temperature sensor and said controller receiving external communications through a transmission module;
a power source in communication with said controller, wherein said controller metes power into said leads and thus to said at least two electrodes, wherein said power source is one of a battery and a rechargeable battery, wherein said battery is one of alkaline, silver nitrate, nickel cadmium, and nickel metal hydride and wherein said rechargeable battery is one of lithium ion, lithium polymer, and a photocell in communication with a capacitor;
a recharge circuit adapted to receive an external signal through said transmission module and to convert the signal into electrical power for delivery to said power source;
an enclosure having said controller, said pressure sensor, said power source, said output circuit, and said recharge circuit within it, said enclosure having a width no more than 13 millimeters and a radius of curvature no less than 12 millimeters;
said controller dispenses through said output circuit a voltage from about 1 volt to about 500 volts and a current from about 10 microamps to about 25 milliamps for a time of application from about 50 microseconds to about 2700 microseconds;
a temperature sensor, and said controller receiving input from said temperature sensor; and,
wherein said enclosure is adapted to install upon the sclera of a mammalian eye and said at least two electrodes are adapted to enter the anterior chamber of a mammalian eye.

20. The electrolysis device of claim 19 wherein said at least two electrodes are one of titanium and platinum.
